SEQUENCE LISTING (34 sequences)

SEQ ID NO: 1 - DNA, 59 bp, artificial
  synthetic primer
gtttttgtttctgaatctttatttttttaagagacaaggtcctctgtgttgctcaggct

SEQ ID NO: 2 - DNA, 41 bp, artificial
  synthetic primer
tgctcaagccactgctctccagcctgagcaacacagaggac

SEQ ID NO: 3 - DNA, 42 bp, artificial
  synthetic primer
ggagagcagtggcttgagcatagccaactgcagtctcgaact

SEQ ID NO: 4 - DNA, 44 bp, artificial
  synthetic primer
aggaggatcatttgagcccaggagttcgagactgcagttggcta

SEQ ID NO: 5 - DNA, 45 bp, artificial
  synthetic primer
cctgggctcaaatgatcctcctgtctcagcttcctgactagctgg

SEQ ID NO: 6 - DNA, 44 bp, artificial
  synthetic primer
gcatggctgtagcctgtagtcccagctagtcaggaagctgagac

SEQ ID NO: 7 - DNA, 54 bp, artificial
  synthetic primer
gactacaggctacagccatgctgcccagctaattaaaaaaaaaaattgtttttc

SEQ ID NO: 8 - DNA, 66 bp, artificial
  synthetic primer
gcaacatagagagacttctgtctctataaaaaggaaaaacaatttttttttttaattagctgggca

SEQ ID NO: 9 - DNA, 56 bp, artificial
  synthetic primer
ctttttatagagacagaagtctctctatgttgcctaggctggtcttgaactcctgg

SEQ ID NO: 10 - DNA, 45 bp, artificial
  synthetic primer
gagatgggaggatcgcctgaggccaggagttcaagaccagcctag

SEQ ID NO: 11 - DNA, 49 bp, artificial
  synthetic primer
cctcaggcgatcctcccatctcccccctagcttttgtgtcaccacattt

SEQ ID NO: 12 - DNA, 50 bp, artificial
  synthetic primer
tgacaggtgggagattgccctggaaatgtggtgacacaaaagctaggggg

SEQ ID NO: 13 - DNA, 44 bp, artificial
  synthetic primer
ccagggcaatctcccacctgtcacccaccaccccctgcatctcc

SEQ ID NO: 14 - DNA, 48 bp, artificial
  synthetic primer
ggagtagtcccatggggacctaggaaaggagatgcagggggtggtggg

SEQ ID NO: 15 - DNA, 49 bp, artificial
  synthetic primer
tttcctaggtccccatgggactactccctgtcccccatgctccaggcac

SEQ ID NO: 16 - DNA, 43 bp, artificial
  synthetic primer
aggtggaggaaggggcagcctgtgcctggagcatgggggacag

SEQ ID NO: 17 - DNA, 54 bp, artificial
  synthetic primer
aggctgccccttcctccacctctctaaaactcaggctgagctatgtacactggg

SEQ ID NO: 18 - DNA, 57 bp, artificial
  synthetic primer
ggggactggatgagatgggcaccacccagtgtacatagctcagcctgagttttagag

SEQ ID NO: 19 - DNA, 54 bp, artificial
  synthetic primer
tggtgcccatctcatccagtcccctgctagtaaccgctagggcttacccgttac

SEQ ID NO: 20 - DNA, 51 bp, artificial
  synthetic primer
ttcccaggtgggcacccgtgggtaacgggtaagccctagcggttactagca

SEQ ID NO: 21 - DNA, 45 bp, artificial
  synthetic primer
ccacgggtgcccacctgggaacaggaggcttggttccacggctgg

SEQ ID NO: 22 - DNA, 45 bp, artificial
  synthetic primer
gccacagcaccctccaccagcccagccgtggaaccaagcctcctg

SEQ ID NO: 23 - DNA, 45 bp, artificial
  synthetic primer
gctggtggagggtgctgtggcacttaccgcatcagcccacagcag

SEQ ID NO: 24 - DNA, 50 bp, artificial
  synthetic primer
gacaggggagagcggatactgccttcctgctgtgggctgatgcggtaagt

SEQ ID NO: 25 - DNA, 47 bp, artificial
  synthetic primer
gaaggcagtatccgctctcccctgtcccctgctatgggcagggcctg

SEQ ID NO: 26 - DNA, 52 bp, artificial
  synthetic primer
gcccagaggtctgacctatttataccccagccaggccctgcccatagcaggg

SEQ ID NO: 27 - DNA, 59 bp, artificial
  synthetic primer
gctggggtataaataggtcagacctctgggccgtccccattcttcccctctctacaacc

SEQ ID NO: 28 - DNA, 59 bp, artificial
  synthetic primer
agatcttgatgaagaagcgctgaggagagagggttgtagagaggggaagaatggggacg

SEQ ID NO: 29 - DNA, 50 bp, artificial
  synthetic primer
ctctctcctcagcgcttcttcatcaagatctggcctcggcggccaagctt

SEQ ID NO: 30 - DNA, 19 bp, artificial
  synthetic primer
aagcttggccgccgaggcc

SEQ ID NO: 31 - DNA, 38 bp, artificial
  synthetic primer
gtttttctttctgaatctttatttttttaagagacaag

SEQ ID NO: 32 - DNA, 19 bp, artificial
  synthetic primer
aagcttggccgccgaggcc

SEQ ID NO: 33 - DNA, 25 bp, artificial
  synthetic primer
gtttttgtttctgaatctttatttt

SEQ ID NO: 34 - DNA, 14 bp, artificial
  synthetic primer
aagcttggccgccg

(19) United States
(12) Patent Application Publication
(10) Pub. No.: US 2012/0122159 A9
(43) Pub. Date: May 17, 2012

(54) PCR-BASED METHOD OF SYNTHESIZING A NUCLEIC ACID MOLECULE

(75) Inventors: Mo-Huang Li, Singapore (SG); Jackie Y. Ying, Singapore (SG); Hongye Ye, Singapore (SG)

(73) Assignee: AGENCY FOR SCIENCE, TECHNOLOGY AND RESEARCH, CONNEXIS (SG)

(21) Appl. No.: 13/140,504

(22) PCT Filed: Dec. 19, 2008
(86) PCT No.: PCT/SG08/00493

§ 371 Date: Jun. 17, 2011

(51) Int. Cl. C12P 19/34; C07H 21/04

(52) U.S. Cl. 435/91.2; 536/23.1

(57) ABSTRACT

There is provided a method of synthesizing a nucleic acid molecule and in one aspect, the method comprises assembling a full length template nucleic acid molecule by PCR in a PCR reaction mixture comprising a set of assembly oligonucleotides having a first average melting temperature and a set of outer amplification primers having a second average melting temperature that is lower than the first average melting temperature, wherein said assembling comprises subjecting the PCR reaction mixture to a first annealing temperature that is higher than the second average melting temperature and; amplifying the full length template nucleic acid molecule by PCR in the PCR reaction mixture wherein said amplifying comprises subjecting the PCR reaction mixture to a second annealing temperature that permits annealing of the outer amplification primers to the full length template nucleic acid molecule.

### FIELD OF THE INVENTION

The present invention relates to PCR methods for synthesizing a nucleic acid molecule.

### BACKGROUND OF THE INVENTION

De novo gene synthesis is a powerful molecular tool for creating and modifying genes. De novo gene synthesis has broad applications including protein engineering (1, 2), development of artificial gene networks (3), and creation of synthetic genomes (4-6). Molecular biology techniques such as gene cloning often involve a PCR step to generate the desired gene, and thus require a DNA template (12). However, natural occurring template DNA is not always available for numerous reasons including lack of access to the relevant source organism, limited environmental or archaeological samples, and degradation of DNA samples or hazards associated with the natural source organism (4). With the ability to synthesize genes de novo in a laboratory, scientists no longer have to rely on the availability and accessibility of natural DNA.

De novo gene synthesis also enables the precise manipulation of genes. By specifying the nucleotide sequence, scientists can readily introduce mutations, incorporate restriction sites for cloning purposes or alter the codon usage to match the known codon preferences of a host cell system (9, 13). Such manipulation can facilitate the study of gene function, structure and expression and improve the expression, localization, detection and purification of proteins as compared to using a template containing a naturally occurring gene sequence (10, 16).

Chemical synthesis of a gene can be achieved through the assembly of multiple oligonucleotides. A longer DNA molecule can be constructed by assembling a pool of oligonucleotides into a larger DNA molecule using a variety of methods including de novo polymerase chain reaction (PCR) (6, 7) or ligase chain reaction (LCR) based (4, 8) synthesis methods. Of the various methods, PCR-based methods appear to be the most efficient and cost-effective (16).

The most reported methods for synthesizing long molecules of DNA are PCR based methods that rely on the use of overlapping oligonucleotides to construct genes. Various methods have been proposed in attempt to optimize the PCR process for long DNA sequences, and to enhance the accuracy of assembly. These methods include the thermodynamically balanced inside-out (TBIO) method (9), successive PCR (10), dual asymmetrical PCR (DA-PCR) (11), overlap extension PCR (OE-PCR) (12, 13), PCR-based two-step DNA synthesis (10, 14, 15), and one-step gene synthesis (16).

The known methods of PCR based gene synthesis often result in the formation of spurious products of higher molecular weights than the desired gene product that reduce the purity of the synthesized products (9-12, 16-19). Furthermore, successful gene synthesis cannot be accurately detected utilizing known PCR based methods, and thus verification of the production of the desired PCR product is typically confirmed by gel electrophoresis. Gel electrophoresis involves manually visualizing the full length PCR products via gel electrophoresis with the help of fluorescence imager. This method involves additional equipment, which is tedious and does not integrate well with lab-on-a-chip methods useful for the development of automated gene synthesis. In addition, gel electrophoresis can only provide end-point analysis of DNA amplification, that is, it can only be used to visualize the DNA products present at the end of the PCR method. PCR amplification of a DNA product is at first stochastic, then exponential and finally stagnant (28).

### SUMMARY OF THE INVENTION

The present invention provides a single reaction method for synthesis of double stranded DNA, including longer DNA molecules that can't practically be synthesized by chemical methods. The method involves synthesizing a gene or nucleic acid molecule by assembling overlapping oligonucleotides and amplifying the assembled product by PCR using a single PCR reaction with distinct oligonucleotides and annealing temperatures for the PCR assembly and amplification processes.

By using a set of assembly oligonucleotides that have a higher average melting temperature than the outer amplification primers used to amplify the desired gene or nucleic acid molecule, the present method reduces the competition between PCR assembly and PCR amplification processes that can occur during conventional one-step PCR-based assembly methods of gene synthesis and thus can provide a more efficient and accurate method for synthesizing long double-stranded genes.

In addition, the present invention provides a method of assembling a full length nucleic acid molecule by real-time PCR (RT-PCR), which may facilitate optimization of reaction conditions for efficient and accurate synthesis of the desired gene product and may permit automated verification and characterization of the gene product that can be readily integrated into a system of automated gene synthesis.

In one aspect, there is provided a method of synthesizing a nucleic acid molecule comprising assembling a full length template nucleic acid molecule by PCR in a PCR reaction mixture comprising a set of assembly oligonucleotides having a first average melting temperature and a set of outer amplification primers having a second average melting temperature that is lower than the first average melting temperature, wherein said assembling comprises subjecting the PCR reaction mixture to a first annealing temperature that is higher than the second average melting temperature and; amplifying the full length template nucleic acid molecule by PCR in the PCR reaction mixture wherein said amplifying comprises subjecting the PCR reaction mixture to a second annealing temperature that permits annealing of the outer amplification primers to the full length template nucleic acid molecule.

In one embodiment, the second annealing temperature is lower than or equal to the second average melting temperature.

In various embodiments, the first average melting temperature may be no less than about 5° C. higher than the second average melting temperature, or may be from about 5° C. to about 25° C. higher than the second average melting temperature.

In various embodiments, the PCR reaction mixture may comprise the set of assembly oligonucleotides at a concentration from about 5 nM to about 80 nM or at a concentration from about 10 nM to about 60 nM.

In various embodiments, the PCR reaction mixture may comprise the set of outer amplification primers at a concentration from about 120 nM to about 1 μM or from about 200 nM to about 800 nM.

In various embodiments, the assembling may comprise conducting from about 5 to about 30 PCR cycles using the first annealing temperature; and the amplifying may comprise conducting from about 10 to about 35 PCR cycles using the second annealing temperature.

In certain embodiments, the full length template may be about 750 base pairs, the assembling may comprise conducting about 15 PCR cycles using the first annealing temperature for the annealing stage, and the amplifying may comprise conducting about 15 PCR cycles using the second annealing temperature. The PCR reaction mixture may comprise the set of assembly oligonucleotides at a concentration of about 10 nM, and may comprise the set of outer amplification primers at a concentration of about 400 nM.

In various embodiments, the PCR may be real-time and the PCR reaction mixture may comprise a fluorescent probe, wherein an increase in fluorescent intensity is linearly proportional to the quantity of the full length template nucleic acid molecule. The fluorescent probe may be LCGreen I. The method may further comprise optimizing the assembling according to fluorescent intensity detected. For example, optimizing may comprise adjusting one or more of (i) time or temperature of denaturing, annealing or elongating; (ii) concentration of the set of assembly oligonucleotides of the set of outer amplification primers; and (iii) number of PCR cycles. The method may be automated.

In another aspect, there is provided a kit comprising a set of assembly oligonucleotides that anneal to form a long double stranded DNA having a gap between adjacent pairs of oligonucleotides and a set of outer amplification primers; wherein the set of assembly oligonucleotides has an average melting temperature that is higher than an average melting temperature of the set of outer amplification primers.

In another aspect, there is provided a method of synthesizing a nucleic acid molecule comprising assembling a full length template nucleic acid molecule by real-time PCR in a PCR reaction mixture comprising a set of assembly oligonucleotides.

As above, the PCR reaction mixture may comprise a fluorescent probe, wherein an increase in fluorescent intensity is linearly proportional to the quantity of the full length template nucleic acid molecule. The fluorescent probe may be LCGreen I. The method may further comprise optimizing the assembling according to fluorescent intensity detected. For example, optimizing may comprise adjusting one or more of (i) time or temperature of denaturing, annealing or elongating; (ii) concentration of the set of assembly oligonucleotides; and (iii) number of PCR cycles. The method may be automated.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention:

FIG. 1. Schematic illustration of the PCR-based method of gene synthesis using oligonucleotide melting temperature variation (“Top Down one-step gene synthesis”). A schematic depiction of one embodiment of the present method, referred to as TopDown (TD) one-step gene synthesis, combines PCR assembly and amplification into a single reaction with different annealing temperatures designed for assembly and amplification. In this embodiment, assembly oligonucleotides and outer amplification primers are designed to have a melting temperature difference of >15° C. to minimize potential interference during PCR.

FIG. 2. Agarose gel electrophoresis results of one-step (30 cycles of assembly/amplification together), TD one-step (40 cycles, 20 assembly followed by 20 amplification), and two-step (PCA: 30 cycles; PCR: 30 cycles) gene synthesis. The TD one-step process involves 20 cycles of PCR assembly performed with an annealing temperature of 67° C., followed by 20 cycles of PCR amplification with an annealing temperature of 49° C., all within a single reaction mixture. The concentrations of assembly oligonucleotides and outer amplification primers are 10 nM and 400 nM, respectively.

FIG. 3. Continuous fluorescence monitoring of real-time PCR gene synthesis with 1× LCGreen I. The first 20 cycles are conducted with an annealing temperature of 67° C., and the next 20 cycles are conducted with an annealing temperature of 49° C. The concentrations of assembly oligonucleotides and outer amplification primers are 10 nM and 400 nM, respectively.

FIG. 4. The assembly oligonucleotide concentration is critical in the successful gene synthesis. S100A4 (752 bp) was synthesized with various assembly oligonucleotide concentrations ranging from 5 nM to 80 nM, and annealing temperatures of 67° C. for the first 20 cycles and 49° C. for the next 20 cycles. (a) Fluorescence as a function of PCR cycle number for oligonucleotide concentrations of 5 nM (⋄), 7 nM (□), 10 nM (Δ), 13 nM (+), 17 nM (x), 20 nM (∘), 40 nM (), 64 nM (▴), and 80 nM (♦). The slopes of fluorescence intensity in the early cycles and cycle 21 to approximately 33 indicate the efficiencies of the assembly and amplification processes, respectively. (b) The corresponding agarose gel electrophoresis results.

FIG. 5. S100A4 (752 bp) is successfully synthesized with various outer amplification primer concentrations ranging from 60 nM to 1 μM. (a) Fluorescence as a function of PCR cycle number for outer primer concentrations of 60 nM (⋄), 120 nM (□), 200 nM (Δ), 300 nM (x), 400 nM (+), and 1 μM (∘). The inset shows the fluorescence signal of the first 20 cycles. (b) The corresponding agarose gel electrophoresis results. The successful synthesis of S100A4 is indicated by the sharp, narrow gel band of the desired length.

FIG. 6. S100A4 is synthesized with various assembly cycles (6-20 cycles), followed by another 20 cycles for amplification. Agarose gel electrophoresis results indicate that full-length assembly is achieved within 11 cycles.

FIG. 7. S100A4 (752 bp) synthesized with various assembly annealing temperatures ranging from 58° C. to 70° C. for the first 20 cycles, followed by an annealing temperature of 49° C. for the next 20 cycles. (a) Fluorescence as a function of PCR cycle number for annealing temperatures of 58° C. (⋄), 60° C. (□), 62° C. (Δ), 65° C. (x), 67° C. (+), and 70° C. (∘). The inset shows the middle 15 cycles (13-27). (b) The corresponding agarose gel electrophoresis results. Higher synthesis yield was obtained with a stringent assembly annealing temperature (>67° C.).

FIG. 8. Concentration effects of SYBR Green I and LCGreen I for TD one-step real-time gene synthesis of S100A4. (a) 0.25× to 5×SYBR Green I. The fluorescence intensity of 1× LCGreen I is also included in this plot for comparison. The fluorescence curves of SYBR Green I are insensitive to the number of PCR cycles, and fail to indicate the DNA length extension during gene synthesis. (b) 0.25× to 5× LCGreen I. The annealing temperatures for assembly and amplification are 58° C. and 49° C., respectively. The concentrations of assembly oligonucleotides and outer primers are 64 nM and 400 nM, respectively.

FIG. 9. The MgSO₄ concentration is critical for successful gene synthesis. (a) Fluorescence of 1× LCGreen I as a function of PCR cycle number for various concentrations of MgSO₄: 1.5 mM (⋄), 2.5 mM (□), 3.0 mM (Δ), 3.5 mM (x), 4.0 mM (), and 5.0 mM (∘). (b) The corresponding agarose gel electrophoresis results. The TD one-step gene synthesis is conducted with annealing temperatures of 58° C. and 49° C. for assembly and amplification, respectively, 1 mM each of dNTP, 10 nM of assembly oligonucleotides, and 400 nM of outer amplification primers. Gene synthesis with 4 mM of MgSO₄ provides the best yield of full-length product.

FIG. 10. Analysis of the products of gene synthesis using RT-PCR and melting peak analysis. (a) Melting peak analyses of the assembled products for S100A4 from one-step and two-step syntheses; two replicas were performed for each oligonucleotides set. The melting curves analyses of assembled genes were acquired using the Roche's LightCycler 1.5 real-time thermal cycling machine with a ramp of 0.05° C./sec for 72-99° C. (b) The corresponding agarose gel electrophoresis results of the assembled products.

Table 1. Data of assembly oligonucleotides.

Table 2. PCR conditions for one-step, two-step, and TD one-step gene synthesis.

Table 3. Some reported optimal gene synthesis conditions.

Table 4. Set of oligonucleotides designed for S100A4.

### DETAILED DESCRIPTION

In PCR-based assembly methods for gene synthesis, a pool of short oligonucleotides is mixed together. Each oligonucleotide contains part of the sequence of either the sense or antisense strand of the desired nucleic acid sequence. In the mixture, oligonucleotides with overlapping complementary sequences anneal to form segments having a double stranded annealed segment and a single stranded overlap segment at one or both ends of the double stranded segment. The end of a strand at the double stranded segment acts as a primer for extension while the single stranded segment acts as a template for the polymerase reaction to create extended double stranded DNA molecules. The extended DNA molecules are then melted and reannealed to form new double/single stranded DNA molecules which then act as new primer/templates which can anneal with other extended complementary template DNA, and generate longer DNA molecules in the next PCR cycle. By repeating this process, the DNA length is gradually increased, and the full length template of the desired sequence is gradually created. The quantity of the assembled full length template DNA is then amplified by a PCR amplification step. Such gene assembly PCR methods can be performed either as a one-step process that combines PCR assembly and PCR amplification in one reaction mixture using a single set of PCR cycles or as a two-step process that involves separate reactions and PCR cycling for the assembly and amplification stages. The one-step gene synthesis process is simple and quick in that it requires only one PCR reaction, but inclusion of the outer amplification oligonucleotides and assembly oligonucleotides together in the same PCR reaction often results in a low yield, and may sometimes fail to produce the desired product. Two-step processes provide better yield of the desired product, but such processes require two distinct PCR reactions, with intervening reagent addition and isolation steps.

As stated above, in previously described one-step PCR-based assembly methods of gene synthesis, outer amplification primers are mixed in the same PCR reaction mixture together with assembly oligonucleotides. The assembly oligonucleotides and amplification primers are commonly designed with homologous melting temperatures to balance the PCR assembly and amplification processes that occur together in the reaction mixture as the PCR progresses. As a result, the outer amplification primers, which are present in excess, tend to preferentially anneal with oligonucleotides that have been extended by the assembly process but which are not full length templates, resulting in a potentially large portion of outer amplification primers participating in the initial gene assembly process, depleting the supply of outer primers available to amplify the full length template once it has been assembled. As well, the supply of deoxynucleotide triphosphates (dNTPs) may be depleted and the PCR reaction may be prematurely halted (17, 21). In addition, internal assembly oligonucleotides which can only be extended in the normal 5′-3′ direction may be inhibitory to the amplification of the full length gene product during the amplification PCR (13). This competitive effect between assembly oligonucleotides and outer amplification primers reduces the yield of the full length gene product and results in the formation of spurious products. This competitive effect is more critical for DNA with high GC content or length (9, 10), and is eliminated in the two-step PCR process whereby the amplification and assembly are performed separately but with the extra cost and effort of fresh PCR mixture and intervening reagent addition and isolation steps.

The present method is based in part on the finding that melting temperature variation can be used to control the efficiencies of the processes of oligonucleotide assembly and full-length template amplification in a single reaction PCR-based method of gene synthesis. Utilizing assembly oligonucleotides and outer amplification primers designed to have different average melting temperatures in a PCR method that includes at least two different annealing temperatures temporally separates the processes of assembly and amplification, and thus reduces the interference between PCR assembly and amplification processes in a single reaction gene synthesis. Thus, the present invention provides a PCR-based method of single reaction gene synthesis that combines the simplicity and cost-effectiveness of known one-step processes with the efficiency of separate assembly and amplification as in known two-step processes.

The present method involves conducting a polymerase chain reaction (PCR) in a single reaction mixture that contains a set of assembly oligonucleotides and a set of outer amplification primers, the set of assembly oligonucleotides having a higher average melting temperature than the set of outer amplification primers. The PCR reaction is conducted in at least two stages, with the first stage using a first annealing temperature that is higher than the average melting temperature of the set of outer amplification primers and that facilitates assembly of the assembly oligonucleotides into a template nucleic acid sequence. The second stage uses a second annealing temperature that permits annealing of the outer amplification primers to the template nucleic acid sequence and amplification of the full length template nucleic acid sequence.

By strategic design of the assembly oligonucleotides and outer amplification primers and selection of suitably different average melting temperatures for the assembly oligonucleotides as compared to the outer amplification primers, it is possible to perform a PCR-based assembly method of gene synthesis as described in the present methods.

Thus, there is provided a method of synthesizing a nucleic acid molecule comprising assembling a full length template nucleic acid molecule by PCR in a PCR reaction mixture comprising a set of assembly oligonucleotides having a first average melting temperature and a set of outer amplification primers having a second average melting temperature that is lower than the first average melting temperature, wherein said assembling comprises subjecting the PCR reaction mixture to a first annealing temperature that is higher than the second average melting temperature and; amplifying the full length template nucleic acid molecule by PCR in the PCR reaction mixture wherein said amplifying comprises subjecting the PCR reaction mixture to a second annealing temperature that permits annealing of the outer amplification primers to the full length template nucleic acid molecule.

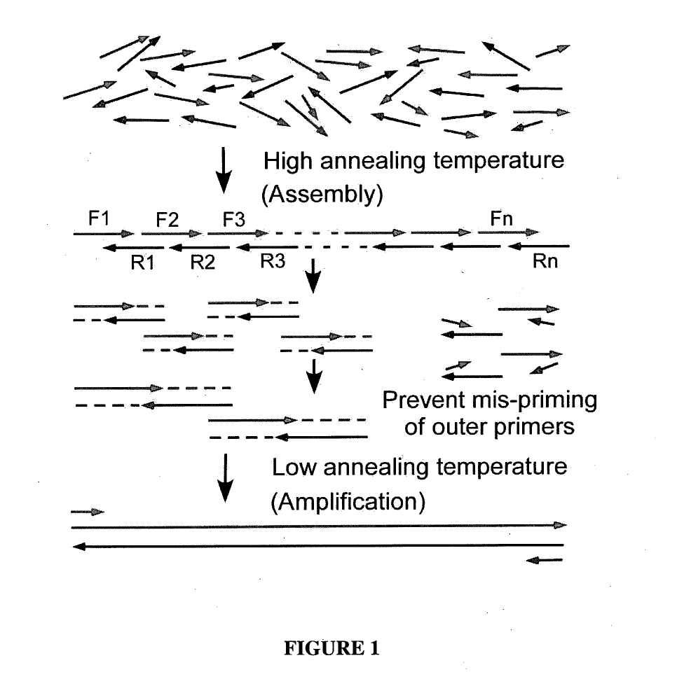

FIG. 1 is a schematic depiction of an embodiment of the present single reaction assembly and amplification PCR method.

PCR methods, conditions and reagents are known in the art (see for example U.S. Pat. Nos. 4,683,195, 4,683,202, and 4,965,188). Generally, PCR amplification is conducted in a PCR reaction mixture that includes a template nucleic acid molecule encoding the sequence that is sought to be amplified, primers designed to anneal to particular complementary target sites on the template, deoxyribonucleotide triphosphates (dNTPS), and a DNA polymerase, all combined in a suitable buffer that allows for annealing of the primers to the template and provides conditions and any cofactors or ions necessary for the DNA polymerase to extend the primer to result in new DNA product.

Briefly, PCR comprises subjecting the PCR reaction mixture to at least one cycle of varying temperatures and for pre-determined times that allow for the stages of denaturing, annealing and elongating. Generally the denaturing, annealing and elongating stages of the PCR cycle each occur at a different specific temperature and it is known in the art to conduct the PCR in a thermal cycler to achieve the required temperature for each step of the PCR cycle. Denaturing is typically performed at the highest temperature to melt any double stranded DNA (either template or amplified product formed in a previous cycle), for example 95° C. if a heat resistant DNA polymerase such as Taq polymerase is used. The annealing stage is performed at a temperature that allows for the oligonucleotides to specifically anneal to a complementary DNA strand, and is typically chosen to facilitate specific annealing while reducing non-specific base pairing. It will be appreciated that the precise annealing temperature chosen depends on the sequences of the oligonucleotides included in the PCR reaction mixture. The elongation stage is performed at a temperature suitable for the particular DNA polymerase enzyme used, to allow the DNA polymerase to synthesize amplified product.

In PCR-based methods of gene synthesis that involve gene assembly, a template nucleic acid molecule is generally not provided in the PCR mixture prior to the commencement of the PCR. Rather, the template is formed during the PCR assembly stage by annealing of the pool of overlapping assembly nucleotides and extension of the overlap by the DNA polymerase to gradually synthesize longer fragments of the desired template, eventually producing a full length unbroken template after a number of PCR cycles, the number of which will depend at least in part on the length of the full length template and the number of overlapping oligonucleotides used to assemble the template.

Thus, in the present methods, it will be appreciated that the PCR reaction mixture includes the necessary components to conduct PCR (including the dNTPs, DNA polymerase and buffer), and that the template and primers are supplied in the initial reaction mixture as the set of assembly oligonucleotides and the set of outer amplification primers, respectively, as described below. It will also be understood that each of assembling and amplifying by PCR as described herein comprises the steps of denaturing, annealing and elongating.

As would be understood, the term “oligonucleotide” refers to a single-stranded nucleic acid molecule comprising at least two nucleotides. The suitable length of an oligonucleotide for use in PCR will be known or can be readily determined. In various embodiments, the length may be from about ten to about one hundred nucleotides. It will be understood by a person skilled in the art that oligonucleotides can be purchased or chemically synthesized by standard known procedures.

The present PCR method involves the use of two types of oligonucleotides in the single PCR reaction mixture: assembly oligonucleotides and outer amplification primers.

A set of assembly oligonucleotides is any group of overlapping oligonucleotides that when annealed together produce a full-length template of a desired nucleic acid sequence or gene but having breaks or gaps along the template on alternating strands of the template, between where one oligonucleotide stops and the next oligonucleotide encoding sequence for the same strand starts. Thus, the set of assembly oligonucleotides is generally designed to cover at least the length of both strands of a double stranded DNA template, such that when all of a complete set of assembly oligonucleotides are annealed together, an annealed double stranded broken template is formed. Each of the assembly oligonucleotides is complementary to either the sense or antisense strand of a portion of a desired nucleic acid sequence or gene and each assembly oligonucleotide partially hybridizes to a least one other assembly oligonucleotide such that when the overlapping assembly oligonucleotides are assembled in the assembly stage of the PCR, a full-length template of the desired nucleic acid sequence or gene is created.

The set of assembly oligonucleotides may be designed to produce a template having a naturally occurring sequence of a gene, or may be designed to introduce mutations or restriction sites into the final template, or to change codons to suit the codon usage of an organism in which the template DNA is ultimately to be expressed. As well, the set of assembly oligonucleotides may be designed to produce novel DNA sequences, such as DNA encoding novel fusion proteins or to insert a tag or DNA target sequence or sequence encoding a protein tag into the template DNA.

A set of outer amplification primers is a group of at least two oligonucleotides that act as primers to anneal to either strand of the full length intact template once assembled from the set of assembly oligonucleotides. The set of outer amplification primers facilitate PCR amplification of all or part of the full length template during the amplification stage of the present methods. In the set of outer amplification primers, at least one primer is complementary to a region at the 3′ end of a coding (or upper) strand of the double stranded full length template and at least one outer amplification primer is complementary to a region at the 3′ end of a complementary (or lower) strand of the double stranded full length template. When hybridized to the full length template in a PCR, the outer amplification primers can facilitate PCR amplification of a selected portion or all of the desired nucleic acid sequence or gene.

An “average melting temperature” refers to the arithmetic mean of the melting temperatures of the oligonucleotides within a set of oligonucleotides, either the assembly oligonucleotides or the outer amplification primers, to which the average melting temperature applies. Thus, the average melting temperature of the assembly oligonucleotides is determined by averaging the melting temperatures of all the assembly oligonucleotides and the average melting temperature of the outer amplification primers is determined by averaging the melting temperatures of all the outer amplification primers. Those skilled in the art will understand the melting temperature of an oligonucleotide to be the temperature at which 50% of a population of that same oligonucleotide will form a stable double stranded helix and the other 50% will be separated into single stranded molecules.

The assembly oligonucleotides and outer amplification primers are designed such that the average melting temperature of the assembly oligonucleotides is higher than the average melting temperature of the outer amplification primers and that the difference in the average melting temperatures is sufficient to reduce the competition between PCR assembly and PCR amplification during single reaction PCR-based gene synthesis. The melting temperature of an oligonucleotide is dependent on various factors including length of the oligonucleotide and the specific nucleic acid sequence of the oligonucleotide, and as such the melting temperatures of each of the assembly oligonucleotides may differ and the melting temperatures of each of the outer amplification primers may differ. However, the oligonucleotides may be designed to minimize the deviation in the melting temperatures of the assembly oligonucleotides and the deviation in the melting temperatures of the outer amplification primers.

The melting temperature for any given oligonucleotide can be calculated using known formulas and known programs, including commercially available software. The use of computer software to design oligonucleotides is known in the art (see for example US Patent Application Pub. No. 2008/0182296, 19). Oligonucleotides can be designed to be optimized for increased gene expression, minimized hairpin formation and homogeneous melting temperatures (9, 19). For example, to design a set of assembly oligonucleotides with minimized deviation between the melting temperatures of each oligonucleotide a computer program may be used which first divides the desired nucleic acid sequence into oligonucleotides of approximately equal lengths by markers, and computes the average and deviation in melting temperatures among the overlapping regions using the nearest neighbour model with Santa Lucia's thermodynamic parameter (23), corrected with salt and oligonucleotide concentrations. The oligonucleotide lengths can then be adjusted through shifting the marker positions to minimize the deviations in the melting temperatures.

Without being limited to any particular theory, it appears that the difference in the average melting temperature of the assembly oligonucleotides and the average melting temperature of the outer amplification primers prevents mis-pairing among the outer amplification primers and the assembly oligonucleotides while facilitating efficient template assembly when an annealing temperature higher than the average melting temperature of the outer amplification primers is used. The difference in average melting temperature should not be too small so as to eliminate any benefit in having different average melting temperatures, while at the same time if the difference is too great, the assembly efficiency may be reduced. When designing primers, it may be advantageous to design the average melting temperature of the outer amplification primers to be greater than about 50° C. in order to enhance specificity.

In some embodiments, the difference in the average melting temperature of the assembly oligonucleotides and the average melting temperature of the outer amplification primers is no less than about 5° C., no less than about 6° C., no less than about 7° C., no less than about 8° C., no less than about 9° C., no less than about 10° C., no less than about 11° C., no less than about 12° C., no less than about 13° C., no less than about 14° C., no less than about 15° C., no less than about 16° C., no less than about 17° C., no less than about 18° C., no less than about 19° C., no less than about 20° C., no less than about 21° C., no less than about 22° C., no less than about 23° C., no less than about 24° C. or no less than about 25° C. In particular embodiments, the difference in the average melting temperature of the assembly oligonucleotides and the average melting temperature of the outer amplification primers is from about 5° C. to about 25° C., from about 7° C. to about 19° C.

A person skilled in the art will recognize that the size of the difference between the average melting temperature of the assembly oligonucleotides and of the outer amplification primers required for successful gene synthesis using the present method will vary depending on the annealing conditions, such as the pH and salt concentration of the PCR mixture, and the specific oligonucleotides. For example, stringent annealing conditions that reduce the likelihood of non-specific oligonucleotide annealing may permit a smaller difference in melting temperatures.

The PCR is conducted in two stages, as described above. The first stage is an assembly stage and comprises one or more cycles of denaturing, annealing and elongating, using an annealing temperature designed to allow for assembly of the set of the assembly oligonucleotides but to reduce annealing of the outer amplification primers to any available complementary nucleic acid molecules that may be present. Specifically, in the assembly stage, the annealing temperature is higher than the melting temperature of the outer amplification primers to permit assembly of the assembly oligonucleotides into the full length template of the desired nucleic acid sequence, while reducing annealing of the outer amplification primers at this stage.

As used herein, the term “annealing temperature” refers to the temperature used during PCR to allow an oligonucleotide to form specific base pairs with a complementary strand of DNA. Typically, the annealing temperature for a particular set of oligonucleotides is chosen to be slightly below the average melting temperature, for example about 1° C., about 2° C., about 3° C. or about 5° C. below, although it may in some instances be equal to or slightly above the average melting temperature for the particular set of oligonucleotides.

For example, the annealing temperature during the assembly stage of gene synthesis may be chosen to be no less than about 5° C., no less than about 6° C., no less than about 7° C., no less than about 8° C., no less than about 9° C., no less than about 10° C., no less than about 11° C., no less than about 12° C., no less than about 13° C., no less than about 14° C., no less than about 15° C., no less than about 16° C., no less than about 17° C., no less than about 18° C., no less than about 19° C., no less than about 20° C., no less than about 21° C., no less than about 22° C., no less than about 23° C., no less than about 24° C. or no less than about 25° C. higher than the average melting temperature of the outer amplification primer set.

The annealing temperature during the assembly stage of gene synthesis may be slightly higher than the average melting temperature of the assembly oligonucleotides. Setting the assembly annealing temperature higher than the average melting temperature of the set of the assembly oligonucleotides may provide several advantages, including: (i) reducing potential competition between the assembly and amplification reactions, (ii) reducing the possibility of truncated oligonucleotides participating in the assembly process and the resulting errors, (iii) providing a more selective annealing condition to reduce the potential for forming secondary structures, and (iv) increasing the specialization of oligonucleotides hybridization, all of which would prevent the generation of faulty sequence, especially for genes with high GC content. It will be appreciated that the extension efficiency of some DNA polymerases is highest at 72° C. and that setting the assembly annealing temperature higher than 72° C. in the present method may reduce the assembly efficiency of the assembly oligonucleotides depending on the DNA polymerase used.

The amplification stage of the PCR is performed using an amplification annealing temperature that permits annealing of the outer amplification primers to the assembled full length template to allow for amplification of some or all of the full length template, depending on where the outer amplification primers are designed to anneal to the template. Generally, the amplification annealing temperature will be closer to the average melting temperature of the outer amplification primers than to the average melting temperature of the assembly oligonucleotides. For example, the amplification annealing temperature may be less than or equal to the average melting temperature of the outer amplification primer set.

As stated above, PCR conditions are generally known in the art. It will be appreciated that the reaction conditions, including for example the oligonucleotide concentration, dNTP concentration, time for each step of a cycle, number of PCR cycles, type of DNA polymerase, pH and the salt concentration of the PCR mixture, required for successful PCR will differ depending on the specific oligonucleotides and polymerase used in the reaction (see for example US Patent Application Pub. No. 2008/0182296). Thus it will be appreciated that the conditions required to achieve successful gene synthesis using the present method will vary depending on the specific assembly oligonucleotides and outer amplification primers used and may need to be optimized for a particular reaction.

DNA polymerases that may be suitable for PCR are known in the art (2, 16, 41-43), including for example Taq DNA polymerase, PFU DNA polymerase, hot start DNA polymerase and ProofStart™ DNA polymerase. In a particular embodiment, the KOD Hot start DNA polymerase (2, 16, 41) is used in the PCR of the present method.

In some embodiments the concentration of the set of assembly oligonucleotides in the PCR reaction mixture required for successful gene synthesis is from about 5 nM to about 80 nM, about 5 nM, about 7 nM, about 10 nM, about 13 nM, about 15 nM, about 17 nM, about 20 nM, about 30 nM, about 40 nM, about 50 nM, about 60 nM or about 80 nM.

In some embodiments, the concentration of the set of outer amplification primers in the PCR mixture is from about 120 nM to about 1 μM, about 120 nM, about 300 nM, about 400 nM, about 500 nM, about 750 nM or about 1 μM.

The number of cycles required for the assembly stage of the PCR will depend at least in part on the number of oligonucleotides, the length of the template to be assembled and the uniformity of the oligonucleotides within the pool. The theoretical minimum number of cycles (x) needed in order to construct a dsDNA molecule of length (L) from uniform oligonucleotide length (n) and overlapping size (s) is given by:

2^{x}n−(2^{x}−1)s>L

In some embodiments, the number of PCR cycles for assembly of the assembly oligonucleotides is from about 5 to about 30 cycles, no less than about 5 cycles, no less than about 6 cycles, no less than about 10 cycles, no less than about 11 cycles, no less than about 15 cycles, no less than about 16 cycles, no less than about 20 cycles, no less than about 25 cycles, or no less than about 30 cycles.

In some embodiments, the number of PCR cycles for the amplification stage for amplification of the full length template is from about 10 to about 35 cycles, no less than about 10 cycles, no less than about 15 cycles, no less than about 20 cycles, no less than about 25 cycles, no less than about 30 cycles, or no less than about 35 cycles.

If desired, the PCR method may begin with a “hot start”, meaning that some reagent is withheld from the reaction mixture which is then incubated at a high temperature, for example 95° C., for a short period of time before addition of the missing reagent. Hot start methods are used to reduce non-specific amplification during the initial set up stages of the PCR by restricting DNA polymerase activity until after the oligonucleotide sample has been heated to or above the oligonucleotides' melting temperature.

As well, if desired, the PCR method may end with a final extended incubation at 72° C. (see for example US Patent Application Pub. No. 2008/0182296).

In one embodiment of the present invention, the PCR method comprises providing 10 nM of assembly oligonucleotides with an average melting temperature of about 65° C. and 400 nM of outside amplification primers with an average melting temperature from about 50 to about 55° C. in a PCR reaction with the following temperature settings: 2 minutes of initial denaturation at 95° C.; followed by 15 cycles of 95° C. for 5 seconds, 67-70° C. for 30 seconds, 72° C. for 30 seconds; followed by 15 cycles of 95° C. for 5 seconds, 50-55° C. for 30 seconds, 72° C. for 30 seconds; and final extension at 72° C. for 10 minutes.

In another embodiment, a 90 second annealing step is provided in the assembly stage of the PCR reaction such that the PCR reaction comprises: 2 minutes of initial denaturation at 95° C.; followed by 15 cycles of 95° C. for 5 seconds, 67-70° C. for 90 seconds, 72° C. for 30 seconds; followed by 15 cycles of 95° C. for 5 seconds, 50-55° C. for 30 seconds, 72° C. for 30 seconds; and final extension at 72° C. for 10 minutes.

The present method may be used to synthesize desired nucleic acid molecules or genes including long and short genes as well as nucleotide molecules encoding part of a gene sequence. The nucleic acid molecules produced using the present method may be used for a variety of purposes including but not limited to the construction of recombinant DNA, optimization of codons for increased gene expression in a particular host, mutation of promoters or transcriptions terminators, and generation of DNA for cell-free or in vitro protein synthesis.

The nucleic acid molecules synthesized by the present methods may be used to express polypeptides or proteins encoded by the synthesized nucleic acid molecules. For example, the nucleic acid sequences synthesized by the present method may be used for recombinant protein expression, construction of fusion proteins and in vitro mutagenesis. Proteins have a wide range of valuable applications in a variety of fields including medicine, pharmaceuticals, research and industry. Standard methods of in vitro protein expression are known in the art. One known method of protein expression, for example, is recombinant protein expression which involves the use of expression vectors, such as plasmids or viral vectors, containing the synthesized nucleic acid sequence to achieve protein expression in an appropriate host cell.

As stated above, the optimal conditions for achieving gene synthesis differ for different oligonucleotides. Factors such as annealing temperature, concentration of oligonucleotides and number of PCR cycles can affect the success of a PCR method, and thus it may be desirable to detect and quantify the synthesized product in order to optimize conditions. To date there has been no means to accurately predict the conditions that will facilitate successful gene assembly by PCR-based methods using a particular set of oligonucleotides. Verification of gene assembly by PCR based-methods is generally done by visualizing the final PCR product using gel electrophoresis. Using this method, verification of gene assembly is delayed until the end of the PCR and the efficiency of gene synthesis after each PCR cycle cannot be determined quantitatively.

Real-time PCR (RT-PCR) is a known technique that involves the use of fluorescence to quantify DNA amplification after each PCR cycle thus permitting continuous monitoring of PCR products throughout the PCR. (28). Generally, for RT-RCR, a PCR reaction is carried out with the addition of a fluorescent marker to the PCR mixture. After each PCR cycle, the level of fluorescence in the mixture is measured to quantify the amount of double stranded DNA product produced. Fluorescent markers that are used for RT-PCR are known in the art including sequence specific RNA or DNA fluorescent probes and double stranded DNA specific dyes (28). RT-PCR is commonly used to monitor gene amplification from template DNA, for example in disease diagnosis (7-8). However to date, RT-PCR has not been used in gene assembly.

The inventors have found that using RT-PCR methods during gene assembly processes allows for optimization of conditions, including the number and length of assembly cycles. Thus, there is further contemplated using real time PCR (RT-PCR) in gene synthesis methods.

Thus there is presently provided a method comprising assembling a full length template nucleic acid molecule by RT-PCR in a PCR reaction mixture comprising a set assembly oligonucleotides. A fluorescent probe may be chosen, such that the fluorescent intensity detected throughout gene assembly is linearly proportional to the length and thus the quantity of full length DNA template molecules.

This method enables optimization of the conditions for PCR-based methods of gene synthesis, verification of the synthesis of the desired nucleic acid molecule or characterization of the synthesized product. Furthermore, the use of RT-PCR enables such optimization, verification and characterization to be integrated into automated methods of gene synthesis.

Thus, by monitoring fluorescent intensity throughout the RT-PCR gene assembly reaction, it is possible to determine the amount of assembled full length DNA template after each cycle and to see the effect of adjusting denaturing, annealing, elongation temperatures, the length of denaturing, annealing, elongation segments of a reaction cycle and the number of cycles performed. In this way, an optimal amount of assembled DNA template may be made.

RT-PCR may be conducted to detect and quantify the products synthesized by PCR-based gene assembly by providing fluorescent markers with particular properties and by optimizing the concentration of such markers. In RT-PCR in gene synthesis, use of a fluorescent marker that binds equally to short and long double stranded DNA molecules results in the fluorescent intensity detected throughout gene assembly being linearly proportional to the length, and thus the quantity, of the full length assembled DNA template molecules.

RT-PCR is commonly conducted using the double stranded DNA specific dye SYBR Green I. However, this dye binds preferentially to long DNA fragments (25, 26) and tends to redistribute from short DNA molecules to longer DNA molecules. During the assembly step of PCR-based gene synthesis, the PCR mixture contains double stranded DNA molecules of various lengths. Thus, during thermal cycling, the SYBR Green I dye bound to shorter pieces of DNA will translocate to the longer DNA molecules as they are synthesized (27), not reflecting accurate results for gene assembly methods. As such, SYBR Green I is not a suitable fluorescent dye for RT-PCR when used in combination with PCR-based methods of gene synthesis. Despite the increase in length of the synthesized DNA molecules, the fluorescent intensity detected using SYBR Green I will remain relatively unchanged throughout the PCR cycles of the assembly step. Thus, RT-PCR has not previously been employed with gene assembly techniques.

The present inventors have found that appropriate fluorescent markers for RT-PCR can be advantageously selected to combine RT-PCR quantification with gene assembly methods in order to optimize the gene assembly PCR methods. The fluorescent markers used to conduct RT-PCR during gene assembly should have a higher affinity for double stranded DNA then single stranded DNA and should not redistribute from short DNA molecules to long DNA molecules during thermal cycling.

Particular fluorescent dyes used to conduct RT-PCR in gene assembly may include for example, LCGreen I (24).

Further, the amount of fluorescent marker used may be optimized to account for the large initial quantity of DNA molecules present in PCR-based methods of gene synthesis, compared to conventional PCR. The initial quantity of DNA molecules present in PCR-based gene synthesis may be larger, by greater than 6 orders of magnitude, than that in conventional PCR amplification methods. The amount of fluorescent dye used to conduct gene synthesis by RT-PCR may be increased to enable detection of synthesized DNA molecules. For example, gene synthesis may be conducted by providing a fluorescent dye, including LCGreen I, at two times the concentration normally provided in standard PCR amplification methods.

By performing PCR gene assembly methods of gene synthesis using RT-PCR, there is provided a method for optimizing gene synthesis. Continuous monitoring of PCR products throughout the assembly and amplification steps facilitates the determination of optimal conditions for gene synthesis for a particular set of oligonucleotides. For example, gene assembly PCR methods performed with RT-PCR may permit the determination of an optimal number of cycles required to complete template assembly, thus enabling the tailoring of the PCR method to reduce unnecessary additional PCR cycling that can result in the production of spurious products (32). In another example, the RT-PCR based methods of gene assembly may be used to determine the optimal annealing temperature for efficient assembly of the assembly oligonucleotides. In addition, RT-PCR gene assembly methods facilitate verification of gene synthesis products after each PCR cycle and thus verification need not be delayed until after the PCR is complete.

Furthermore, when gene synthesis is performed using RT-PCR, the synthesized products may be characterized by DNA melting curve analysis. DNA melting curve analysis, in combination with RT-PCR and DNA melting simulation software (31, 39), can be used to estimate the purity and quantity of PCR products. Methods of performing DNA melting curve analysis are known in the art (25) and generally involve detecting the level of fluorescence while slowly heating a PCR product in order to determine the melting temperature. As each double stranded DNA has its own specific melting temperature, it will be understood by one skilled in the art that successful gene synthesis using the present method would yield a product with a single, sharp melting peak, while incomplete synthesis would result in a broad melting curve. In addition, the integrated area of the melting peak in the negative derivative of the fluorescence with respect to temperature would give the quantity of the desired full-length product (38).

RT-PCR eliminates the need for manual visualization using gel electrophoresis to verify gene synthesis and to quantify and characterize the synthesized products. Thus using RT-PCR in gene synthesis permits the use of automated methods for optimizing gene synthesis and verifying and characterizing synthesized products. For example, optimization of the number of cycles in the gene assembly step may be automated such that when a level of fluorescence indicative of assembly of the full nucleic acid molecule of the desired sequence is detected the thermocycler automatically switches to the amplification step of gene synthesis. The level of fluorescence indicative of complete assembly of a particular nucleic acid molecule may be pre-determined using RT-PCR. In another example, melting curve analysis, facilitated by the use of RT-PCR, can be performed by automated methods such as a computer program thus enabling automated characterization of synthesized products that can be readily integrated into systems of automated gene synthesis including for example, lab-on-a-chip methods (U.S. Provisional Application 60/963,673).

As described above, RT-PCR may be applied in the present single reaction mixture PCR-based method of gene synthesis. Furthermore, the method of RT-PCR described herein may also be used in other PCR-based method of gene synthesis. For example, RT-PCR may be used to optimize and automate the known one-step and two-step PCR-based methods of gene synthesis.

Also contemplated are kits and commercial packages that combine a set of amplification oligonucleotides and a set of outer amplification primers as described above, the outer amplification primers having an average melting temperature lower than the average melting temperature of the set of the assembly oligonucleotides.

The present methods are further exemplified by way of the following non-limited examples.

### Examples

### Materials and Methods

Design of Oligonucleotides for Gene Synthesis

Gene sequence for the promoter of human calcium-binding protein A4 (S100A4, 752 bp; chrl:1503312036-1503311284) (22) was selected for synthesis via assembly PCR. Assembly oligonucleotides were derived by a custom-developed program, which first divided the given sequence into oligonucleotides of approximately equal lengths by markers, and computed the average and deviation in melting temperatures among the overlapping regions using the nearest neighbour model with Santa Lucia's thermodynamic parameter (23), corrected with salt and oligonucleotide concentrations. Next, the oligonucleotide lengths were adjusted through shifting the marker positions to minimize the deviations in the overall overlapping melting temperature. The summary of the assembly oligonucleotide set is shown in Table 1 with the detail information provided in Table 4.

Non-Competitive One-Step Real-Time Gene Synthesis

Non-competitive one-step process was optimized using real-time PCR conducted with Roche's LightCycler 1.5 real-time thermal cycling machine with a temperature transition of 20° C./s. Real-time gene synthesis was conducted with 20 μl of reaction mixture including 1×PCR buffer (Novagen), 1 μl of 0.25× to 5×SYBR Green I (1×=1/20,000 dilution; Invitrogen) or LCGreen I (Idaho Technology Inc.), 4 mM of MgSO₄, 1 mM each of dNTP (Stratagene), 500 μg/ml of bovine serum albumin (BSA), 5-80 nM of assembly oligonucleotides, 60 nM-1 μM of outer amplification primers, and 1 U of KOD Hot Start (Novagen). The PCR were conducted under the following conditions: 2 min of initial denaturation at 95° C.; 20 cycles of 95° C. for 5 s, 58-70° C. for 30 s, 72° C. for 30 s; followed by 20 cycles of 95° C. for 5 s, 49° C. for 30 s, 72° C. for 30 s; and final extension at 72° C. for 10 min. Desalted oligonucleotides were purchased from Research Biolabs (Singapore) and Proligo (Singapore) without additional purification.

One-Step and Two-Step PCR-Based Gene Synthesis

Conventional gene synthesis via PCR was performed either as a one-step process, combining PCR assembly and amplification into a single stage, or as a two-step process with separate stages for assembly and amplification. All PCR reactions, whether for assembly or amplification, were run in standard 0.2-ml PCR tubes with a commercial thermal cycler (DNA Engine PTC-200, Bio-Rad) using the same assembly oligonucleotides set and outer amplification primers as in the non-competitive one-step PCR. The one-step process was performed with 50 μl of reaction mixture including 1×PCR buffer (Novagen), 4 mM of MgSO₄, 1 mM each of dNTP (Stratagene), 500 μg/ml of BSA, 10 nM of assembly oligonucleotides, 400 nM of outer amplification primers, and 1U of KOD Hot Start (Novagen). The one-step PCR was conducted under the following conditions: 2 min initial denaturation at 95° C.; 30 cycles of 95° C. for 5 s, 58° C. for 30 s, 72° C. for 30 s; and final extension at 72° C. for 10 min. The PCR protocol of the two-step process was essentially the same as that for one-step process except for the concentration of oligonucleotides and annealing temperature. For PCR assembly, 10 nM of assembly oligonucleotides were used without outer amplification primers. For gene amplification, 2 μl of the assembled product was diluted in 25 μl of amplification reaction mixture with outer amplification primers at a concentration of 400 nM each, and an annealing temperature of 49° C. was employed. The PCR conditions of the three types of gene synthesis are summarized in Table 2. Some reported optimal gene synthesis conditions are set out in Table 3.

Agarose Gel Electrophoresis

The synthesized products were analyzed by 1.5% agarose gel (NuSieve® GTG®, Cambrex Corporation), stained with ethidium bromide (Bio-Rad Laboratories) or SYBR Green (Invitrogen), and visualized using Typhoon 9410 variable imager (Amersham Biosciences). Gel electrophoreses were performed at 100 V for 45 min with 100 bp ladder (New England) and 5 μl of DNA samples.

Results

Performance of TD One-Step Gene Synthesis

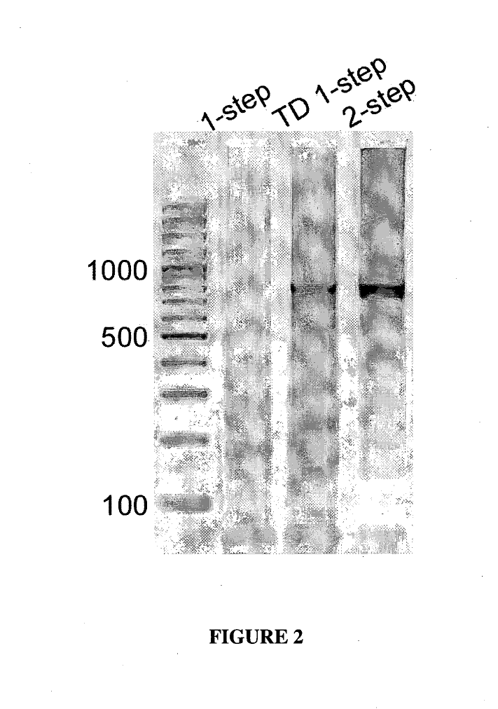

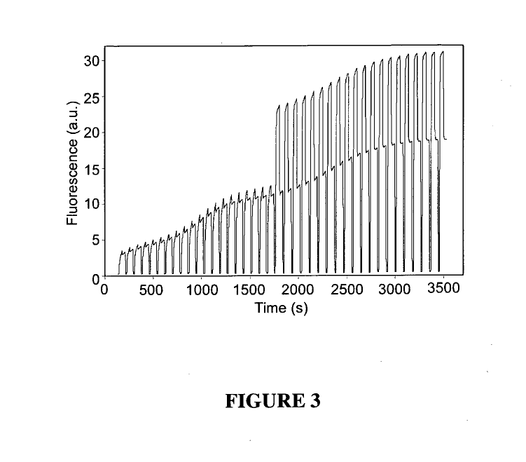

Successful gene synthesis was achieved using TD one-step process and the conventional two-step process, while no obvious full-length gene product was obtained in the conventional one-step PCR process, as shown by gel electrophoresis (FIG. 2). The TD one-step process was conducted with an annealing temperature (Tₐₕ) of 67° C. (average Tₘ of assembly oligonucleotides=66° C.) for the first 20 cycles, followed by an annealing temperature of 49° C. (average Tₘ of outer amplification primers=50.1° C.) for another 20 cycles. The continuous fluorescence monitoring revealed the efficiency of the gene synthesis process (FIG. 3). Unlike the exponential nature of PCR amplification, the assembly efficiency was more likely linear in nature.

Performance of Gene Synthesis Using Real-Time Gene Synthesis

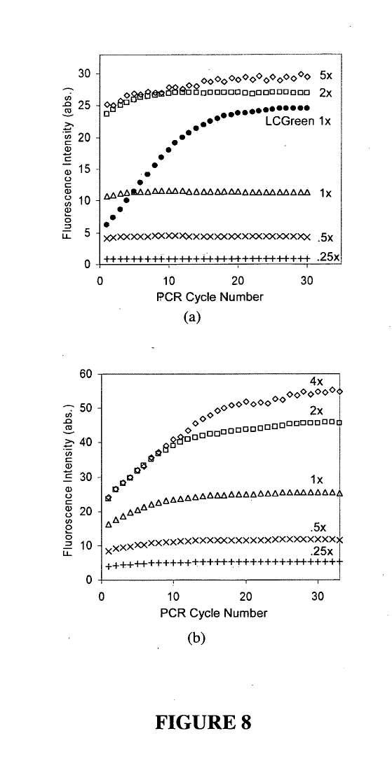

Two intercalating fluorescent dyes (SYBR Green I and LCGreen I) were investigated for real-time gene synthesis (FIG. 8). The LCGreen I (24) was more suitable for studying the real-time gene synthesis. LCGreen I has a fluorescence spectrum similar to the commonly adopted SYBR Green I in real-time PCR and is compatible with most real-time thermal cyclers. The SYBR Green I binds preferentially to long DNA fragments (25, 26), and redistributes from short DNA molecules to long DNA molecules during thermal cycling (27). This makes it difficult to analyze the fluorescence signal since the PCA mixture contains various lengths of dsDNA. The fluorescent intensity remains unchanged during the PCR as shown in FIG. 8(a). In contrast, the use of LCGreen I provides a fluorescent intensity curve that demonstrates the increase in number and the extension in length of the synthesized DNA molecules as the assembly reaction proceeds (see FIG. 8(b)).

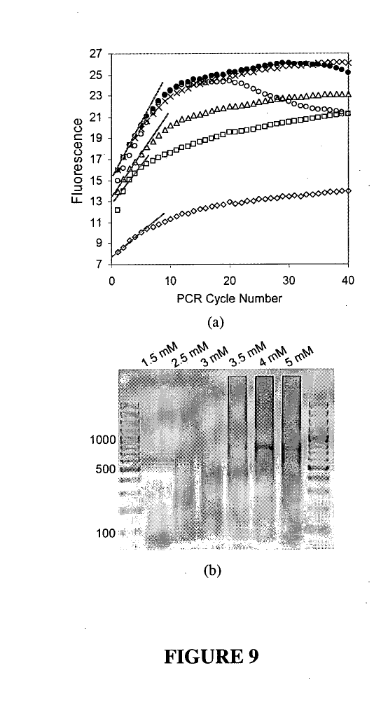

The initial quantity of DNA molecules (˜6 pmol; 10 nM×20 μl×30 oligonucleotides) in the PCA mixture was much larger, by >6 orders of magnitude, than that in standard PCR amplification (<10⁶ copies of template DNA) (28). The real-time PCR conditions were adjusted for this factor. The optimal concentration of LCGreen I was studied and increased to 2 times the concentration used in standard PCR (FIG. 8). The dNTPs concentration was adjusted from 0.2 mM each for standard PCR to 1 mM each to prevent the depletion of dNTPs. The Mg²⁺ ion (MgSO₄) concentration was empirically optimized (at 4 mM) based on the concentration of dNTP, which could chelate with Mg²⁺ and affect the polymerase activity (29, 30). (FIG. 9). The manufacturer's recommended Mg²⁺ ion concentration was 1.5 mM for standard PCR with 0.2 mM of dNTPs each.

Analysis of Real-Time Gene Synthesis

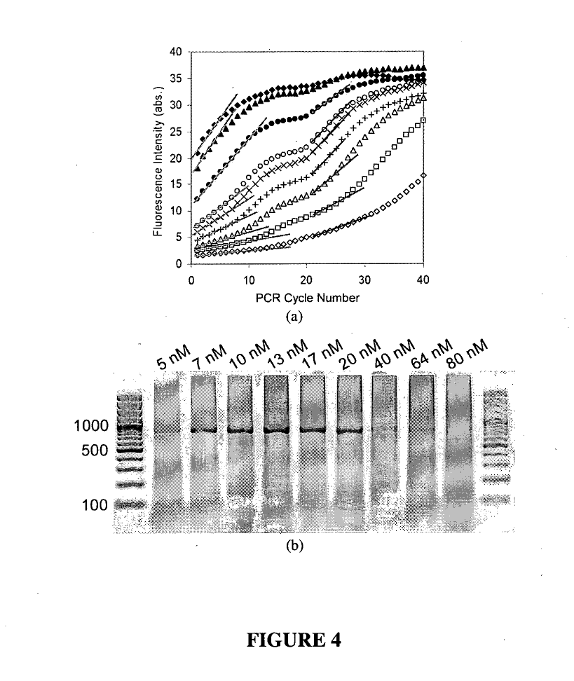

Mechanistically, gene synthesis took place in several phases, as revealed by the variation in slopes with the number of PCR cycles (FIG. 4). This phenomenon was remarkable with an assembly oligonucleotide concentration of 10-20 nM. In the early cycles of PCA, most annealing between paired oligonucleotides formed an extendable duplex, which could undergo extension by polymerase (phase 1; cycles<7). The fluorescence signal revealed a linear increment of DNA length extension with each cycle. In contrast to that reported by Wu et al. (16) and Lee et al. (21), the assembly efficiency increased with further PCR cycles (phase 2; cycles-7-14). Our hypothesis was that the assembly process switched in favor of full-length template amplification as the full-length fragments emerged, and was promoted by the excess outer primers. The PCA reaction then reached the first plateau (phase 3; cycles 15-20) whereby the outer primers priming was limited by the elevated annealing conditions (Tₐₕ-Tₘ=15° C.). At cycle #21, the annealing temperature was reduced to 49° C. to match with the Tₘ of primers (phase 4; cycles—21-29). The exponential amplification was boosted, and caused a sudden jump in fluorescence signals. Finally, the process reached the second plateau, presumably due to the depletion of outer primers or non-specific products annealing (phase 5). The plateau stages were delayed or completely missing for low oligonucleotide concentration (<7 nM) due to its low assembly efficiency.

For gene synthesis with >64 nM of assembly oligonucleotides, the PCR process reached the plateau within 15 cycles. Additional cycles would most likely favor non-specific PCR, and lead to the generation of spurious bands and the build-up of high molecular weight products in gel electrophoresis (FIG. 4b), as observed in most reported gene synthesis results (9-12, 16-19). The consistent gel results and real-time PCR curves suggested that the optimal assembly oligonucleotide concentration was 10-20 nM for TD gene synthesis, which coincided with that of both the one-step (16, 17) and two-step (18) processes.

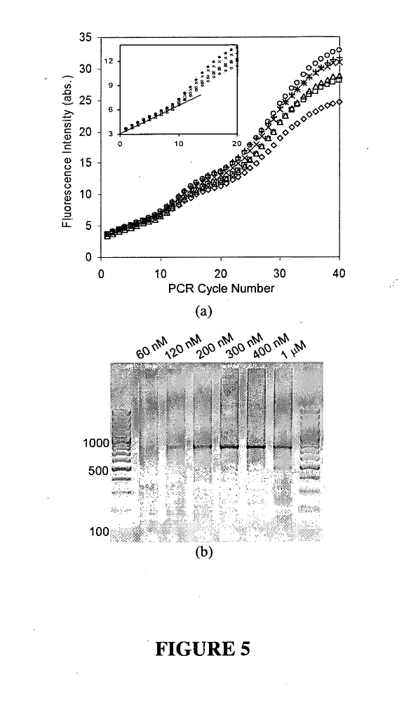

The effect of outer amplification primers was further investigated by varying the outer amplification primer concentration from 60 nM to 1 μM while keeping the assembly oligonucleotide concentration at 10 nM. The highest full-length quantity was obtained with 400 nM of outer amplification primers (FIG. 5), which was consistent with observations in one-step (16) and two step (18) processes. Assembly efficiencies, depicted by the slopes of fluorescence increment, were indifferent in the early cycles (<cycle 7), even though the outer amplification primer concentration was varied by 16-fold (inset in FIG. 5a). This demonstrated the non-interference feature of the TD process, wherein the outer amplification primers did not intervene with the assembly process. The assembly efficiencies started to deviate at around cycle 8 as the full-length products emerged, in favor of full-length template amplification. Unlike the assembly oligonucleotide concentration, which dominated the assembly reaction and critically influenced the success of gene synthesis, the outer amplification primer concentration was less critical. It presumably controlled the late amplification process and the quantity of desired DNA. The optimal PCR cycles depended on the initial assembly oligonucleotide concentration and target gene length. This was clearly demonstrated by the experiment on assembly oligonucleotide concentration (FIG. 4). As assembly oligonucleotide concentration increased from 20 nM to 80 nM, the full-length band gradually disappeared and became widened.

The overlapping assembly was a parallel process. Relatively few PCR cycles were needed to complete the assembly. The theoretical minimum number of cycles (x) needed in order to construct a dsDNA molecule of length (L) from uniform oligonucleotide length (n) and overlapping size (s) is given by:

2^{x}n−(2^{x}−1)s>L

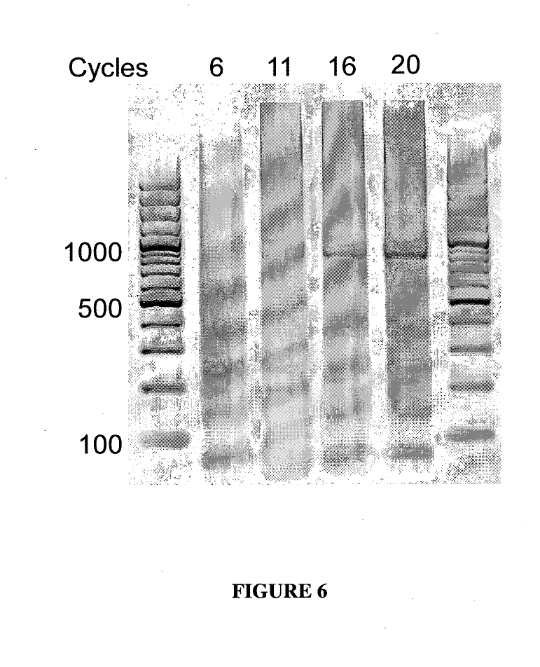

Theoretically, six PCA cycles were sufficient for assembling S100A4 (752 bp) from a pool of 40mer oligonucleotides with an overlap of 20 nucleotides. To determine whether excess cycling was necessary for gene assembly, the optimal conditions determined in previous experiments were used with various PCA cycles of 6-20, followed by 20 amplification cycles. Gene synthesis was fairly efficient. Indeed, full-length assembly was achieved within 11 PCA cycles (FIG. 6).

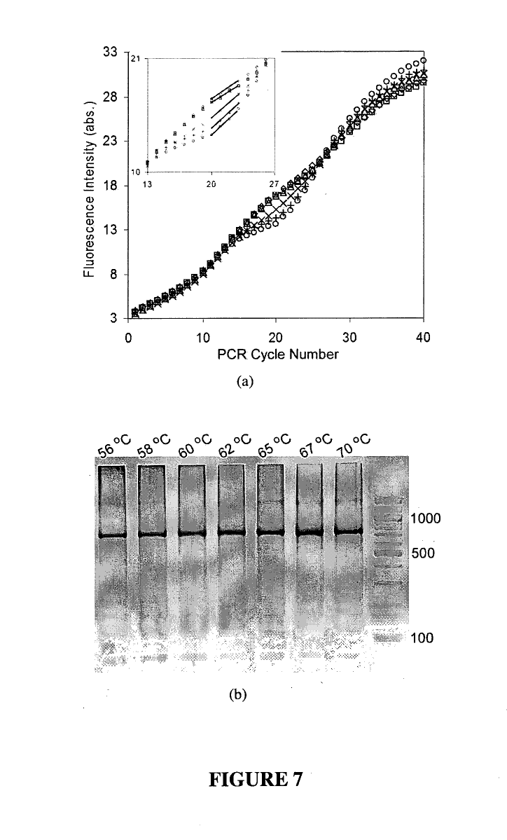

The gene synthesis was insensitive to the variation in assembly annealing temperature (Tₐₕ) from 58° C. to 70° C., as visualized in both gel results and fluorescence signals (FIG. 7). The fluorescence intensity curves were indiscriminate to the annealing temperatures during the assembly phase (first 13 cycles), and began to deviate only after the first phase (see inset in FIG. 7a). The indifference in fluorescence intensity during the first 13 cycles implied that the outer amplification primers did not intervene with the assembly reaction. The outer amplification primers were designed with an average Tₘ of 50.9° C., which meant that the primers encountered an annealing stringent of 7.1-19.9° C. (Tₐₕ-Tₘ) during the PCA process. This suggested that the melting temperature window (ΔTₘ of primers and oligonucleotides) could potentially be reduced to 7.1° C., and ensure the non-competitive feature of TD gene synthesis method. Interestingly, a higher yield of the desired DNA was obtained with a stringent annealing temperature (>67° C.) higher than the average Tₘ of the assembly oligonucleotides (66° C.).

Melting Curve Analysis of Synthesized DNA Molecules

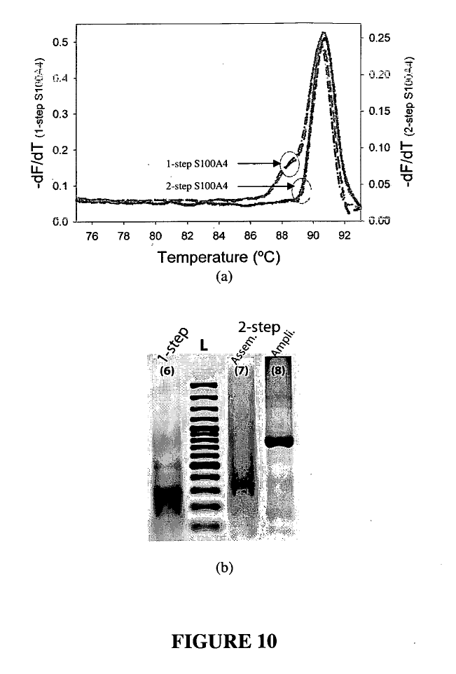

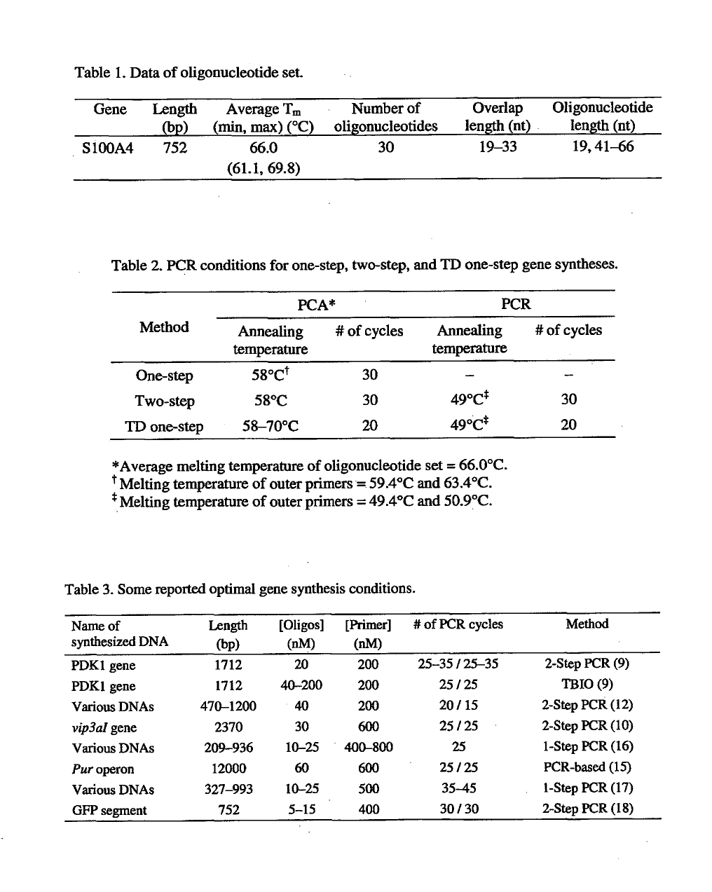

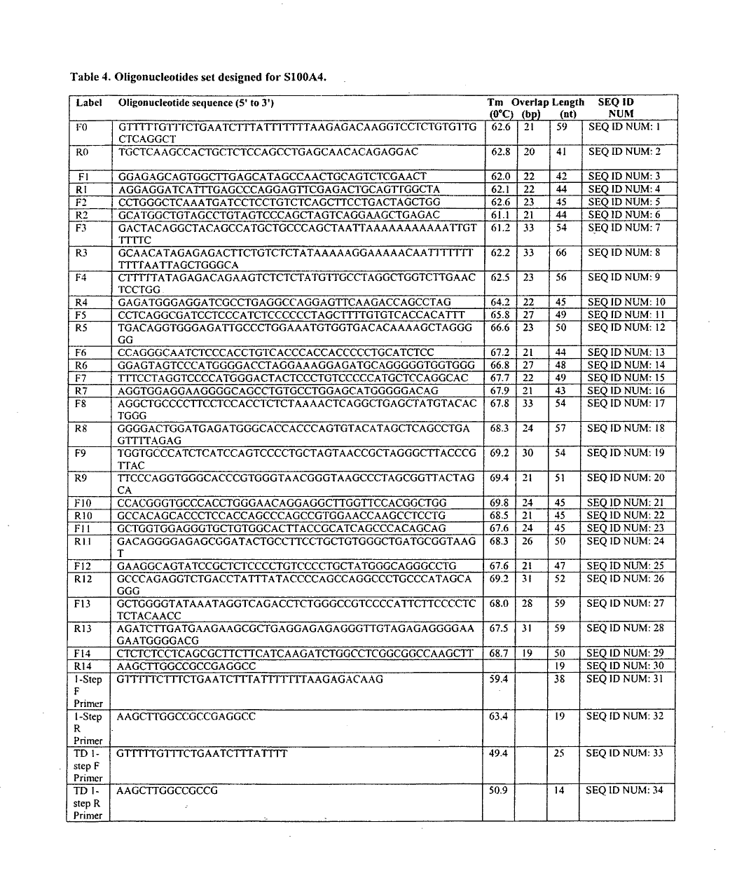

Melting curve analysis was conducted on products synthesized using RT-PCR in the conventional one-step and two-step PCR-based assembly methods of gene synthesis (FIG. 10). Successful synthesis generated a single, sharp melting peak in the melting curve, which corresponded to a distinct band in gel electrophoresis for the two-step process. In contrast, for the one-step process, the melting curve was broad, indicating that the product was a mixture of DNA molecules with intermediate lengths, as reflected in the smeared gel electrophoresis. The majority of one-step products were incomplete products with lengths of approximately 200-300 base pairs.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Concentrations given in this specification, when given in terms of percentages, include weight/weight (w/w), weight/volume (w/v) and volume/volume (v/v) percentages.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs.

### REFERENCES

1. Ramachandran, N., Hainsworth, E., Bhullar, B., Eisenstein, S., Rosen, B., Lau, A. Y., Walter, J. C. and Labaer, J. (2004) Self-assembling protein microarrays. Science, 305, 8690
2. Cox, J. C., Lape, J., Sayed, M. A. and Helling a, H. W. (2007) Protein fabrication automation. Protein Sci., 16, 379-390.
3. D. Sprinzak and M. B. Elowitz (2005) Reconstruction of genetic circuits. Nature, 438, 443448.
4. Smith, H. O., Hutchison, C. A., III, Pfannkoch, C. and Venter, J. C. (2003) Generating a synthetic genome by whole genome assembly: (ΦX174 bacteriophage from synthetic oligonucleotides. Proc. Natl. Acad. Sci. USA, 100, 15440-15445.
5. Gibson, D. G., Benders, G. A., Andrews-Pfannkoch, C., Denisova, E. A., Baden-Tillson, H., Zaveri, J., Stockwell, T. B., Brownley, A., Thomas, D. W., Algire, M. A., Merryman, C., Young, L., Noskov, V. N., Glass, J. I., Venter, J. e., Hutchison, c. A., Ill, and Smith, H. O. (2008) Complete chemical synthesis, assembly, and cloning of a Mycoplasma genitalium genome. Science, 319, 1215-1220.
6. Cello, J., Paul, A V. and Wimmer, E. (2002) Chemical synthesis of poliovirus cDNA: Generation of infectious virus in the absence of natural template. Science, 297, 1016-1018.
7. Kodumal, S. J., Patel, K. G., Reid, R., Menzella, H. G., Welch, M. and Santi, D. V. (2004) Total synthesis of long DNA sequences: Synthesis of a contiguous 32-kb polypeptide synthase gene cluster. Proc. Natl. Acad. Sci. USA, 101, 15573-15578.
8. Au, L. C., Yang, F. Y., Yang, W. J., Lo, S. H. and Kao, C. F. (1998) Gene synthesis by a LCR-based approach: High-level production of leptin-L54 using synthetic gene in Escherichia coli. Biochem. Biophys. Res. Commun., 248, 200-203.
9. Gao, X., Yo, P., Keith, A, Ragan, T. J. and Harris, T. K. (2003) Thermodynamically balanced inside-out (TBIO) PCR-based gene synthesis: A novel method of primer design for high-fidelity assembly of longer gene sequences. Nucleic Acids Res., 31, e143.
10. Xiong, A-S., Yao, Q.-H., Peng, R.-H., Li, X., Fan, H.-Q., Cheng, Z.-M. and Li, Y. (2004) A simple, rapid, high-fidelity and cost-effective PCR-based two-step DNA synthesis method for long gene sequences. Nucleic Acids Res., 32, e98.
11. Sandhu, G. S, Aleff, R. A. and Kline, B. C. (1992) Dual asymmetric PCR: One-step construction of synthetic genes. Biotechniques, 12, 14-16.
12. Young, L. and Dong, Q. (2004) Two-step total gene synthesis method. Nucleic Acids Res., 32, e59.
13. Prodromou, C. and Pearl, L. (1992) Recursive PCR: A novel technique for total gene synthesis. Protein Eng., 5, 827-829.
14. Stemmer, W. P., Crameri, A, Ha, K. D., Brennan, T. M. and Heyneker, H. L. (1995) Single-step assembly of a gene and entire plasmid from large numbers of oligodeoxyribonucleotides. Gene, 164, 49-53.
15. Xiong, A-S., Yao, Q.-H., Peng, R.-H., Duan, H., Li, X., Fan, H.-Q., Cheng, Z.-M. and Li, Y. (2006) PCR-based accurate synthesis of long DNA sequences. Nat. Protoc., 1, 791-797.
16. Wu, G., Wolf, J. B., Ibrahim, A. F., Vadasz, S., Gunasinghe, M. and Freeland, S. J. (2006) Simplified gene synthesis: A one-step approach to PCR-based gene construction. J. Biotech., 124, 496-503.
17. Kong, D. S., Carr, P. A, Chen, L., Zhang, S, and Jacobson, J. M. (2007) Parallel gene synthesis in a microfluidic device. Nucleic Acids Res., 35, e61.
18. Huang, M. C., Ye, H., Kuan, Y. K., Li, M.-H. and Ying, J. Y. (2008) Integrated two-step gene synthesis in a microfluidic device. Lab Chip, in press.
19. Hoover, D. M. and Lubkowski, J. (2002) DNA Works: An automated method for designing oligonucleotides for PCR-based gene synthesis. Nucleic Acids Res. 30, e43.
20. Wu, G., Dress, L. and Freeland, S. J. (2007) Optimal encoding rules for synthetic genes: The need for a community effort. Mol. Syst. Biol., 3, 1-5.
21. Lee, J. Y., Lim, H.-W., Yoo, S.-I., Zhang, B.-T. and Park, T. H. (2005) Efficient initial pool generation for weighted graph problems using parallel overlap assembly. Lect. Notes Camp. ScL, 3384, 215-223.
22. Saleem, M., Kweon, M.-H., Johnson, J. J., Adhami, V. M., Elcheva, I. et al. (2007) S100A4 accelerates tumorigenesis and invasion of human prostate cancer through the transcriptional regulation of matrix metalloproteinase 9. Proc. Natl. Acad. Sci. USA, 103, 14825-14830.
23. Santa Lucia, J., Jr. and Hicks, D. (2004) The thermodynamics of DNA structural motifs. Annu. Rev. Biophys. Biomol. Struct., 33, 415-440.
24. Herrmann, M. G., Durtschi, J. D., Bromley, L. K., Wittwer, C. T. and Voelkerding, K V. (2006) Amplicon DNA melting analysis for mutation scanning and genotyping: Cross-platform comparison of instruments and dyes. Clin. Chem., 52, 494-503.
25. Wittwer, C. T., Reed, G. H., Gundry, C. N., Vandersteen, J. G. and Pryor, R J. (2003) High-resolution genotyping by amplicon melting analysis using LCGreen. Clin. Chem., 49, 853860.
26. Giglio, S., Monis, P. T. and Saint, C. P. (2003) Demonstration of preferential binding of SYBR Green I to specific DNA fragments in real-time multiplex PCR Nucleic Acids Res., 31, e136.
27. Varga, A. and James, D. (2006) Real-time PCR and SYBR Green I melting curve analysis for the identification of plum pox virus strains C, EA, and W: Effect of amplicon size, melt rate, and dye translocation. J. Viral. Methods, 132, 146-153.
28. Wittwer, C. T., Herrmann, M. G., Moss, A A and Rasmussen, R P. (1997) Continuous fluorescence monitoring of rapid cycle DNA amplification. BioTechniques, 22, 130-138.
29. Ely, J. J., Reeves-Daniel, A., Campbell, M. L., Kohler, S, and Stone, W. H. (1998) Influence of magnesium ion concentration and PCR amplification conditions on cross-species PCR BioTechniques, 25, 38-40.
30. von Ahsen, N., Wittwer, C. T. and Schütz, E. (2001) Oligonucleotide melting temperatures under PCR conditions: Nearest-neighbor corrections for Mg²⁺, deoxynucleotide triphosphate, and dimethyl sulfoxide concentrations with comparison to alternative empirical formulas. Clin. Chem., 47, 1956-1961.
31. Rasmussen, J. P., Saint, C. P. and Monis, P. T. (2007) Use of DNA melting simulation software for in silico diagnostic assay design: Targeting regions with complex melting curves and confirmation by real-time PCR using intercalating dyes. BMC Bioinformatics, 8, 107-118.
32. Luo, R and Zhang, D. (2007) Partial strands synthesizing leads to inevitable aborting and complicated products in consecutive polymerase chain reactions (PCRs). Sci. China Ser. C Life Sci., 50, 548.
33. Bell, D. A. and DeMarini, D. M. (1991) Excessive cycling converts PCR products to random-length higher molecular weight fragments. Nucleic Acids Res., 19, 5079.
34. Binkowski, B. P., Richmond, K E., Kaysen, J., Sussman, M. R and Belshaw, P. J. (2005) Correcting errors in synthetic DNA through consensus shuffling. Nucleic Acids Res., 33, e55.
35. Carr, P. A., Park, J. S., Lee, Y. J., Yu, T., Zhang, S, and Jacobson, J. M. (2004) Protein-mediated error correction for de novo DNA synthesis. Nucleic Acids Res., 32, e162.
36. Fuhrmann, M., Oertel, W., Berthold, P., Hegemann, P. (2005). Removal of mismatched bases from synthetic genes by enzymatic mismatch cleavage. Nucleic Acids Res., 33, e58.
37. Don, R. H., Cox, P. T., Wainwright, B. J., Baker, K, Mattick, J. S. (1991) ‘Touchdown’ PCR to circumvent spurious priming during gene amplification. Nucleic Acids Res., 19, 4008.
38. Ririe, K M., Rasmussen, R P. and Wittwer, C. T. (1997) Product differentiation by analysis of DNA melting curves during the polymerase chain reaction. Anal. Biochem., 245, 154160.
39. Blake, R D., Bizzaro, J. W., Blake, J. D., Day, G R, Delcourt, S. G., Knowles, J., Marx, K A and Santa Lucia, J., Jr. (1999) Statistical mechanical simulation of polymeric DNA melting with MELTSIM. Bioinformatics, IS, 370-375.
40. Neuzil, P., Pipper, J. and Hsieh, T. M. (2006) Disposable real-time microPCR device: Lab-on-a-chip at a low cost. Mol. BioSyst., 2, 292-298.
41. Mamedov, T. G., Padhye, N. V., Viljoen, H. and Subramanian, A. (2007) Rational de novo gene synthesis by rapid polymerase chain assembly (PCA) and expression of endothelial protein-C and thrombin receptor genes. J. Biotech., 131, 379-387.
42. Arezi, B., Xing, W., Sorge, J. A. and Hogrefe, H. H. (2003) Amplification efficiency of thermostable DNA polymerase. Anal. Biochem., 321, 226-235.
43. Cherry, J., Nieuwenhuijsen, B. W., Kaftan, E. J., Kennedy, J. D. and Chanda, P. K. (2008) A modified method for PCR-directed gene synthesis from large number of overlapping oligodeoxyribonucleotides. J. Biochem. Biophys. Methods, 70, 820-822.

1. A method of synthesizing a nucleic acid molecule comprising:
assembling a full length template nucleic acid molecule by PCR in a PCR reaction mixture comprising a set of assembly oligonucleotides having a first average melting temperature and a set of outer amplification primers having a second average melting temperature that is lower than the first average melting temperature, wherein said assembling comprises subjecting the PCR reaction mixture to a first annealing temperature that is higher than the second average melting temperature and;
amplifying the full length template nucleic acid molecule by PCR in the PCR reaction mixture wherein said amplifying comprises subjecting the PCR reaction mixture to a second annealing temperature that permits annealing of the outer amplification primers to the full length template nucleic acid molecule.

2. The method according to claim 1 wherein the second annealing temperature is lower than or equal to the second average melting temperature.

3. The method according to claim 1 wherein the first average melting temperature is no less than about 5° C. higher than the second average melting temperature.

4. The method according to claim 1 wherein the first average melting temperature is from about 5° C. to about 25° C. higher than the second average melting temperature.

5. The method according to claim 1 wherein the PCR reaction mixture comprises the set of assembly oligonucleotides at a concentration from about 5 nM to about 80 nM.

6. The method according to claim 1 wherein the PCR reaction mixture comprises the set of assembly oligonucleotides at a concentration from about 10 nM to about 60 nM.

7. The method according to claim 1 wherein the PCR reaction mixture comprises the set of outer amplification primers at a concentration from about 120 nM to about 1 μM.

8. The method according to claim 1 wherein the PCR reaction mixture comprises the set of outer amplification primers at a concentration from about 200 nM to about 800 nM.

9. The method according to claim 1 wherein said assembling comprises conducting from about 5 to about 30 PCR cycles using the first annealing temperature.

10. The method according to claim 1 wherein said amplifying comprises conducting from about 10 to about 35 PCR cycles using the second annealing temperature.

11. The method according to claim 1 wherein the full length template is about 750 base pairs, said assembling comprises conducting about 15 PCR cycles using the first annealing temperature for the annealing stage, and said amplifying comprises conducting about 15 PCR cycles using the second annealing temperature.

12. The method according to claim 11 wherein the PCR reaction mixture comprises the set of assembly oligonucleotides at a concentration of about 10 nM.

13. The method according to claim 11 wherein the PCR reaction mixture comprises the set of outer amplification primers at a concentration of about 400 nM.

14. The method according to claim 1 wherein the PCR is real-time PCR.

15. The method of claim 14 wherein the PCR reaction mixture comprises a fluorescent probe and wherein an increase in fluorescent intensity is linearly proportional to the quantity of the full length template nucleic acid molecule.

16. The method of claim 15 wherein the fluorescent probe is LCGreen I.

17. The method according to claim 1 further comprising optimizing said assembling according to fluorescent intensity detected.

18. The method of claim 17 where said optimizing comprises adjusting one or more of:
a. time or temperature of denaturing, annealing or elongating;
b. concentration of the set of assembly oligonucleotides or the set of outer amplification primers; and
c. number of PCR cycles.

19. The method of claim 14 wherein the method is automated.

20. A kit comprising a set of assembly oligonucleotides that anneal to form a long double stranded DNA having a gap between adjacent pairs of oligonucleotides and a set of outer amplification primers; wherein the set of assembly oligonucleotides has an average melting temperature that is higher than an average melting temperature of the set of outer amplification primers.

21. A method of synthesizing a nucleic acid molecule comprising assembling a full length template nucleic acid molecule by real-time PCR in a PCR reaction mixture comprising a set of assembly oligonucleotides.

22. The method of claim 21 wherein the PCR reaction mixture comprises a fluorescent probe and wherein an increase in fluorescent intensity is linearly proportional to the quantity of the full length template nucleic acid molecule.

23. The method of claim 22 wherein the fluorescent probe is LCGreen I.

24. The method of claim 21 further comprising optimizing said assembling according to fluorescent intensity detected.

25. The method of claim 24 wherein said optimizing comprises adjusting one or more of
a. time or temperature of denaturing, annealing or elongating;
b. concentration of the set of assembly oligonucleotides; and
c. number of PCR cycles.

26. The method of claim 21 wherein the method is automated.
